(19) United States
(12) Patent Application Publication
(10) Pub. No.: US 2006/0067988 A1
(43) Pub. Date: Mar. 30, 2006

(54) Pharmaceutical formulations designed to supplement the diet of anorexic patients

(76) Inventors: Claudio Franco Omini, Paullo (IT); Giuseppe Zuccari, Paullo (IT)

Correspondence Address: YOUNG & THOMPSON, 745 SOUTH 23RD STREET, 2ND FLOOR, ARLINGTON, VA, 22202, US

(21) Appl. No.: 11/236,585

(22) Filed: Sep. 28, 2005

(30) Foreign Application Priority Data
Sep. 29, 2004 (IT) MI2004A001849

(51) Int. Cl. A61K 38/17; A61K 47/00

(52) U.S. Cl. 424/439; 514/2

(57) ABSTRACT

The present invention relates formulations designed to supplement the diet of patients who are anorexic, otherwise debilitated or convalescent, or suffer from other eating disorders, comprising casein hydrolysates and proteins derived from whey in the ratio of 1:1 and 1:5 in weight respectively.

### SUMMARY OF INDUSTRIAL INVENTION

This invention relates to pharmaceutical formulations for the preparation of diet supplements suitable for the nutrition of patients who are anorexic or otherwise debilitated or convalescent, or suffer from other eating disorders. Said formulations are characterised by the presence of proteins deriving from whey, casein hydrolysates, carbohydrates and polyunsaturated fatty acids, with the addition of micronutrients including vitamins and mineral salts, especially calcium salts.

### DESCRIPTION OF THE INDUSTRIAL INVENTION

Eating disorders comprise a set of pathological conditions which have been encountered increasingly often by doctors in various specialist fields in the last few decades. Eating disorders generally correspond to behavioural modifications and mental distress which require the involvement of psychologists and psychiatrists. In parallel with psychological treatment, dietary measures are also required, which involve administering to the patient diet supplements that provide sufficient metabolic energy and micronutrients to ensure correct maintenance of the normal physiological functions of the human body. Said diet supplements must necessarily be formulations with pleasant organoleptic characteristics which provide a sufficient source of energy. Moreover, these formulations must induce the least possible feeling of fullness. These formulation characteristics are also required for diet supplements administered to patients who are debilitated or for any other reason require a calorie, multivitamin and salt intake additional to their usual diet. Indeed, also this type of patient generally has a conflictual relationship with food.

Numerous diet supplements can provide a high energy intake, including the family of diet foods marketed under the Resource (Novartis Medical Nutrition), Meritene (Novartis Consumer Health), Fortimel (Nutricia) and Ensure (Abbott) brands, etc. However, none of these products seems to be specifically designed for anorexic patients who, as already stated, have very different diet supplementation requirements from other patients, who only need a high energy intake. The peculiarity of anorexic patients is evident because, in addition to compulsive behaviour towards food which has a large psychological component, they also present manifest physical complications. Among these complications, those affecting the immune system, the activity of which is reduced and compromised, are particularly evident and generalised. A formulation suitable for use as a diet supplement must therefore also contain substances which increase the defensive responses of the immune system.

Anorexic patients also have a premature feeling of fullness, so in order to be acceptable to them, a high-protein diet supplement must necessarily induce a feeling of fullness at the latest possible stage. All these limitations require the design of a formulation which meets said needs with the smallest possible number of active constituents.

A protein intake is essential to supplement the diet of anorexic patients. As this intake can be effectively provided by various types of food proteins of vegetable or animal origin, it is advantageous to use protein originating from soya, corn, rice, etc. The animal proteins most commonly used are those deriving from milk and egg white. The milk proteins must commonly used can be generically divided into casein and whey proteins. The latter proteins are obtained from cow's milk after precipitation of casein at pH 4.6, and contain a varied group of proteins, the main ones being β-lactoglobulin (2-4 g/L), ═-lactoalbumin (0.6-1.7 g/L), albumin (0.2-0.4 g/L) and immunoglobulin (0.5-1.8 g/L) (Dairy Chemistry and Physics University of Guelph www.foodsci.uoguelph.ca, 2004). Whey proteins are preferred for the purposes of this invention, because they provide a high intake of calorie and nutrient content. These proteins also show a marked immunomodulating action (Bounous et al., Clin. Invest. Med. 12:154-61; 1989). This immunostimulating capacity of whey is far superior to that obtained with casein or soya protein (Bounous et al., J. Nutr. 113: 1415-21; 1983). The proteins obtained from whey are therefore particularly indicated in the diet of anorexic patients, and are consequently particularly preferred for the purposes of this invention. However, there is a serious limitation on the use of whey proteins for anorexic patients because, as demonstrated by Hall W L et al. (Br. J. Nutr. 89:239-48; 2003), their administration induces a greater feeling of fullness greater than casein. This serious drawback has been overcome in the present invention by associating, whey proteins with those deriving from casein hydrolysis. In cow's milk, the proportions of protein deriving from casein account for approx. 80% of the total protein, while those deriving from whey correspond to approx. 20% of the total (Dairy Chemistry and Physics University of Guelph www.foodsci.uoguelph.ca, 2004). Surprisingly, it has been demonstrated in the present invention that reversal of the ratio present in cow's milk, ie. a casein to whey protein ratio of between 1:1 and 1:5, produces an adequate energy and nutritional intake for anorexic patients, and also minimises the feeling of fullness undeniably caused by the intake of food proteins. Casein is known to contain peptide fragments which can have an agonistic activity towards the opioid receptors (Teshemecher H et al., Biopolymers 43:99-117; 1997) and also cause behavioural changes (Goody R J and Kitchen I, JPET 296:744-8; 2000). It is interesting to note that stimulation of the opioid receptors induces hyperphagia, as demonstrated nearly 80 years ago by Flowers et al. (Proc Soc Biol Med 26: 572-4; 1929). More recently, other authors have extended these first experimental findings, indicating that stimulation of the opioid receptors participates in the neuronal mechanisms that govern preferences in the choice of food type (Znang N et al., JEPET 285: 908-14; 1998). These authors demonstrated that stimulation of the opioid receptors in the central nervous system leads patients to prefer more palatable foods, especially those rich in fats. The association of the present diet supplements with foods of any origin, and in particular with high-fat foods, to promote weight gain in anorexic patients, is therefore also claimed in this invention. Associations of the diet supplements claimed in this invention with smoothies, especially those containing bananas, walnuts and all fruits and nuts rich in edible vegetable fats, are particularly preferred for this purpose.

Further, if required, this invention also claims the concomitant use of L-glutamine or dietary peptides containing L-glutamine to enhance the immunostimulating action of whey proteins. L-glutamine is known to increase numerous functional parameters of the immune system (Newsholme P et al., Braz J Med Biol Res 36: 153-63; 2003). The administration of L-glutamine also has many other favourable effects on the human body, with the result that its use in diet supplements leads to a marked improvement in the general conditions of critical patients, and promotes the convalescence of patients who have undergone surgical operations or suffered injuries.

The doses of L-glutamine particularly preferred in this invention are those which determine activation of the immune system, namely between 0.3 and 30 g/day, depending on the severity of the disorder.

If required, in addition to mineral salts and vitamins, especially those of Group B, the formulations claimed in this invention may be enriched with calcium salts such as Ca lactate, Ca carbonate, Ca gluconate and all possible pharmaceutically acceptable calcium salts.

### EXAMPLES

The following examples are given by way of example but not of limitation of the claims contained herein.

### Example 1

### Granulate Formulation for Monodose Sachets

Whey protein 3.670 g—Caseinates 3.0 g—Maltodextrins 13.330 g—Fatty acids 10.00 g (of which linoleic acid 2.9 g; linolenic acid 0.35 g)—Glutamine 1.00 g—Thiamine HCl 0.253 mg—Riboflavin 0.28 mg—Pyridokine HCl 0.33 mg—Folic acid, Na salt 0.055 mg—Vit. B12 (1:1000) 0.55 mg—Ca pantothenate 1.18 mg—Niacin Na 3.3 mg—Biotin Na 0.0103 mg—Na ascorbate 13.75 mg—Resveratrol 200—mg Vit. A palmitate (500 IU) 0.62 mg—Vit. D3 0.000913 mg—Vit. E acetate (50%) 4.4 mg—Vit. K 0.01284 mg—Se proteinate (0.2%) 1.66 mg—Iron gluconate (ferrous) 20 mg—NaI 0.04 mg—NaF 0.38 mg—Mn gluconate 4.8 mg—ZnO 2.325 mg—Cu gluconate 3.6 mg —Cr picolinate 0.0403 mg—MoNa₂O₄ H₂O 0.0126 mg—MgO 51.8 mg—Ca lactate 710 mg—Na₂CO₃ 95 mg—Acesulfame-K 40 mg—Aerosol 200 150 mg—orange, carrot and lemon flavouring 600 mg.

### Example 2

A mixture of caseinates and whey proteins qualitatively similar to that reported in example 1 was tested on 6 healthy adult volunteers (4 M, 2 F) aged between 40 and 60 years with a BMI (body mass index), calculated according to the weight/height² formula, of under 30.

Each volunteer fasted for at least 12 hours before receiving the formulation studied, dissolved in 150 ml of mineral water. Immediately before taking the formulation under study, volunteers rated their feeling of hunger on a visual analogue scale (10 mm) 30 min. after taking the formulation under study, volunteers rated their feeling of desire for food, again on a visual analogue scale (10 mm). In all cases the visual analogue scale ranged from a minimum of 0 to a maximum of 10.

The formulations studied were:
a) whey protein only (6.67 g)
b) as stated in example 1, with whey protein (g 3.67) and caseinates (3.0 g)
c) caseinates only (6.67 g)

All volunteers received all the treatments, which were blinded, once a day. The results are shown in the table below:

TABLE 1

| | Feeling of hunger/desire for food | | |
|---|---|---|---|
| | M + SD | | |
| Treatment | a | b | c |
| before | 7.8 + 1.027 | 7.92 + 0.793 | 7.93 + 0.819 |
| after | 7.33 + 0.907 | 8.1 + 0.721 | 8.05 + 0.792 |
| t-test | 0.006 | 0.116 | 0.017 |

The results clearly demonstrate that the association of whey protein and caseinates (treatment b) did not significantly reduce the feeling of hunger/desire for food in the volunteers tested, unlike the administration of whey protein alone (treatment a).

1. Formulations designed to supplement the diet of patients who are anorexic, otherwise debilitated or convalescent, or suffer from other eating disorders, comprising casein hydrolysates and proteins derived from whey in the ratio of between 1:1 and 1:5 in weight respectively.

2. Formulations as claimed in claim 1, containing glutamine in the quantity of between 0.3 and 30 g/day in weight.

3. Formulations as claimed in claim 2, wherein glutamine is also present in peptide form.

4. Formulations as claimed in claim 1, containing carbohydrates and polyunsaturated fatty acids with the addition of micronutrients comprising vitamins and mineral salts, especially calcium salts.

5. Formulations as claimed in claim 4, wherein the polyunsaturated fatty acids are linolenic and linoleic acid.

6. Formulations as claimed in claim 1, which can be administered in association with foods of any origin, and in particular foods rich in edible vegetable fats, to promote weight gain.

7. Formulations as claimed in claim 1, which can be administered in association with smoothies or other drinks or edible fluids.

8. A method of treatment of anorexic patients which involves the administration of casein hydrolsates and proteins derived from whey in the ratio of between 1:1 and 1:5 in weight respectively.

9. Formulations as claimed in claim 2, containing carbohydrates and polyunsaturated fatty acids with the addition of micronutrients comprising vitamins and mineral salts, especially calcium salts.

10. Formulations as claimed in claim 3, containing carbohydrates and polyunsaturated fatty acids with the addition of micronutrients comprising vitamins and mineral salts, especially calcium salts.
